(19) United States
(12) Patent Application Publication
(10) Pub. No.: US 2012/0021467 A1
(43) Pub. Date: Jan. 26, 2012

(54) METHOD OF PRODUCING XYLITOL AND ARABINOSE AT SAME TIME FROM HEMICELLULOSE HYDROLYSATES

(75) Inventors: Hourui Zhang, Fujian (CN); Xiangxiang Qin, Fujian (CN); Alhua Cai, Fujian (CN); Yuheng Zhou, Fujian (CN); Haishan Chen, Fujian (CN)

(73) Assignee: Thomson Biotech (Xiamen) PTE. LTD., Fujian (CN)

(21) Appl. No.: 12/841,309

(22) Filed: Jul. 22, 2010

(51) Int. Cl. C12P 19/02

(52) U.S. Cl. 435/105

(57) ABSTRACT

The present invention relates to a method of producing xylitol and arabinose at same time from hemicellulose hydrolysates, material rich in xylan is hydrolyzed by acid or enzymes to obtain hydrolysate mostly containing xylose and arabinose, then inoculated in Issatchenkia orientalis S-7 and/or Issatchenkia occidentalis LJ-3 to eliminate the inhibitor of microbes, after that/or at the same, inoculated a yeast of Candida tropicalis which can consume the glucose, transform the xylose to xylitol and can not consume the arabinose, and fermented, then fermented liquor containing xylitol and arabinose is obtained. After separation, xylitol with purity more than 99% and arabinose with high purity are obtained. After concentration and crystallization respectively, crystalline xylitol and crystalline arabinose are obtained respectively.

### FIELD OF THE INVENTION

The present invention relates to biotechnology, more particularly, to applications for preparing xylitol of a candida tropicalis yeast, and medicine and health-caring application of xylitol with high purity.

### BACKGROUND OF THE INVENTION

Hemicellulose is the polysaccharide except the cellulose in cell wall of plant, the content of hemicellulose can be various according to different plant, mostly, the content is about 20-35% WT of the plant, is it is the richest polysaccharide in the earth except the cellulose.

Cellulose is homo-polysaccharide only consisted by glucose unit, in contrast, hemicellulose is a hetero-polysaccharide consisted by main chain consisted of xylose and branch chains consisted of arabinose, mannose and galactose. The xylose main chain of hemicellulose usually is consist by 50-150 xylose units, there is no crystal structure and chains are fixed to the surface of the microfibre of the cellulose.

Hemicelluloses are much easily hydrolyzed than celluloses, in conditions under 100 to 300° C. and in diluted acid, the hemicelluloses are easily hydrolyzed into hydrolysate mostly consisted of pentose. Producing chemicals such as xylitol, ethanol, etc. by fermentation of the hemicellulose hydrolysate is an efficiency path to use the mass hemicellulose resource.

Although the process that the hemicelluloses being hydrolyzed into hydrolysate mostly consisted of pentose in diluted acid is not difficult, however, series microbe metabolic inhibitor will be brought in the hydrolysis. If the fermentation property of the hemicelluloses hydrolysate is to be improved, the microbe metabolic inhibitor must be removed, namely, the detoxification is necessary.

Dozens of the toxic ingredients have been identified in hemicelluloses hydrolysate, representative toxic ingredients comprises three types, i.e. furfural type compounds, such as furfural, 5-(Hydroxymethyl) furfural; fatty acid type compounds such as formic acid, acetic acid, propionic acid; a series of compounds containing benzene ring, such as guaiacol, benzaldehyde, ferulic acid.

In prior reports, vacuum evaporation, solvent extraction, activated carbon adsorption, macroporous resin adsorption, or ion-exchange resin can remove some toxic ingredients and improve the fermentation performance of hydrolysates. However, one physical or chemical method is can only remove a type of toxic ingredient, usually plurality of physical and chemical measures are needed to treat the hemicelluloses hydrolysate to get well detoxification effect, but this will be high cost.

Some microbes in natural can degrade some toxic ingredients of the hemicelluloses hydrolysate, for example, white-rot fungi capable of degrading lignin (LIN Hai; LU Gang; ZHANG Qingna etc. Screening and identification of strain degrading lignin and its application in paper-making wastewater treatment, Journal of University of Science and Technology Beijing, 2007, 29(6): 569-573; TAO Yang; LIAO Jun-he etc. Progress of Making Pulp by White-rot Fungi, Journal of Cellulose Science and Technology, 2007, 15(1): 70-74; bacteria and yeast capable of degrading furfural, 5-(Hydroxymethyl) furfural (DAI Shu-ling, ZHANG Lu-jia, Progress of Biodegradation (Biotransformation) of Furfural and Its Derivatives, Amino Acids & Biotic Resources, 2007, 29(4): 41-45; Liu ai-ping etc. Recent progress of ethanol production from lignocellulose by yeasts, Letters In Biotechnology, 2004, 15(2): 193-196), sac fungi capable of degrading fatty acid (Gangqiang He, Guocheng DU etc. Cutinase Production from Short-chain Organic Acids by Thermobif' uta fusca, Chinese Journal of Biotechnology, 2008, 24(5): 821-828).

Recently, enzymes have been used to attempt to remove the toxic ingredients. Jonsson etc. removed most monoaromatic phenolic compounds in the acid hydrolysate of wood by adding laccase and peroxidase, after treating, the hydrolysate is used for ethanol ferment, as a result, the glucose consuming rate and the ethanol production rate is rise to five times. (Jonsson J L, Palmqvist E, Nilvebrant N O., Detoxification of wood hydrolysates with laccase and peroxidase from the white-rot fungus Trametes versicolor. Appl. Microbiol. Biotechnol. 1998, (49):691-697)), but complicated toxic components need complicated enzyme system, this will greatly add the detoxification cost.

Lopez etc. ferment the lignocellulose hydrolysates by Conichaeta ligniaria NRRL 30616, not only the furfural and 5-(Hydroxymethyl) furfural are removed obviously, but also the phenols are removed is obviously. After treating, the hydrolysates is used for ethanol ferment, 1.66% ethanol is produced in 80 h, while the no-treated hydrolysates have not any ethanol (Lopez J M, Nichols N N, Dien B S et al. Isolation of microorganisms for biological detoxification of lignocellulosec hydrolysates. Appl Microbiol Biotechnol. 2004, 64 (1):125-131).

These researches shows that degrade the complicated toxic components in the lignocelluloses hydrolusates by microbes are doable. In particularly, if a metabolic system which can degrade the toxic components of three types. i.e. a microbe which can degrade the furfural type compounds, fatty acid type compounds and compounds containing benzene ring, and the microbe will not consume the xylose, then if the hemicelluloses hydrolysates is treated by the microbe, the disadvantages of detoxification in prior art that complicated progress, high cost will be overcome, and this is friend to circumstance.

In addition, hemicellulose is easy to be hydrolyzed into hydrolysates containing xylose, arabinose and the other saccharides by diluted acid or enzyme. Only being recovered respectively, the xylose and the arabinose can be the commercially used.

Pure xylose is mainly used to produce xylitol. The xylitol is deoxidized by xylose and has same sweetness, and no-cariogenicity, in metabolism it need not the help of insulin, and will no case rapid change of the blood sugar, thus it has important application in anticarious food and diabetics food.

Pure arabinose has similar taste to cane sugar, and the other special functions has attract attentions. For example, arabinose can inhibit the activity of the sucrase, thus restrain human body to absorb the cane sugar, thus can control the blood sugar rise caused by eating sugar; arabinose also can inhibit the activity of lipase, thus can be used to prevent obesity. In addition, arabinose can be used to be medicine intermediate for composing nucleotide medicine and can be widely used in medicine industry.

However, whether the xylose or arabinose, only when they have high purity, they can be high value in commerce. Thus the isolations of the xylose and the arabinose are very important in production.

U.S. Pat. No. 6,086,681 (Lindroos, et al. Method for recovery of xylose from solutions”, 2000) relates to a method for the recovery of xylose from xylose-containing aqueous solutions containing 30-60% by weight of xylose on dissolved solids, in which method the solution is treated to produce a solution supersaturated with xylose, xylose is crystallized from the supersaturated solution and the xylose crystals are recovered. But the invention did not relate to how to recover the xylose remained in the mother liquor, or how to recover the arabinose in the aqueous solutions.

U.S. Pat. No. 6,872,316 (Recovery of xylose, 2005) relates to a process of producing a xylose solution from a biomass hydrolysate by subjecting the biomass hydrolysate to nanofiltration and recovering as the nanofiltration permeate a solution enriched in xylose. Because the xylose can more easily permeate the nanofiltration membrane, the other impurities such as oligosaccharide, hexose, cytochromes are remained in the other side of nanofiltration membrane, so the purity of xylose after nanofiltration can be improved to at least twice than the original solution. In this invention, the arabinose is easy to permeate the nanofiltration membrane as the xylose, thus it cannot separate the xylose from the arabinose. In addition, the nanofiltration has limited effect on recovering the monose.

Positive ion is able to adsorb the monosaccharide (or furfuryl alcohol), recovering different monosaccharide or furfuryl alcohol by material containing the positive ion is one of the ways to recover the target monosaccharide or furfuryl alcohol from mixture solution containing multi-monosaccharides.

U.S. Pat. No. 6,506,897 (Method of preparing L-arabinose from sugar beet pulp, 2003) relates to a method of preparing crystalline L-arabinose by extraction of sugar beet pulp, from which sugar has been extracted, in a strong alkaline solution, by hydrolysis of the obtained crude araban with a strong acid at an elevated temperature, by neutralization and filtration of the obtained solution, by chromatographic separation of the L-arabinose fraction, by purification of the obtained L-arabinose solution by means of cation and anion exchangers and adsorbent resins, and by recovering the pure L-arabinose as a crystalline product. Obviously, the method that firstly extract in a strong acid, then obtained crude araban with a strong acid is complicated, and the invention did not show whether there is xylose in the hydrolysate or not and did not show how to recovery the xylose.

China application 01816511.7 (PCT/FI2001/000848, recovering a monosaccharide from a solution using a weakly acid cation exchange resin is for the chromatographic separation, Applicants: DANISCO SWEETENERS OY) relates to a method for recovering a monosaccharide selected from the group consisting of rhamnose, arabinose, xylose and mixtures thereof from a solution containing the same by a multistep process using chromatographic separation comprising at least one step, where a weakly acid cation exchange resin is used for the chromatographic separation. Both the description and the figures of the invention show that the xylose and the arabinose are not separated completely. And the invention did not show how to selectively convert the saccharide into furfuryl alcohol and how to separate the saccharide and the furfuryl alcohol.

Besides the cation exchange resin for the chromatographic separation of saccharide, some inorganic materials containing the positive ion are used for recovering the saccharide.

U.S. Pat. No. 4,664,718 (Process for separating arabinose from a pentose/hexose mixture, 1987) related to a process for the liquid phase adsorptive separation of arabinose from an aqueous feed mixture of monosaccharides containing arabinose along with other aldopentose and aldohexoses. The feed is contacted with a calcium-Y or calcium-X type zeolite. But the invention did not relate to recover the xylose or xylitol.

U.S. Pat. No. 4,857,642 (Process for separating arabinose from a mixture of other is aldoses, 1989) relates to a process for the liquid phase adsorptive separation of arabinose from an aqueous feed mixture of monosaccharides containing arabinose along with other aldoses and ketoses. The feed is contacted with an ammonium X-type zeolite. In U.S. Pat. No. 4,880,919 (Kulprathipanja, Process for separating arabinose from a mixture of aldoses, 1989), arabinose is separated from mixtures of monosaccharides containing arabinose and other aldopentoses and aldohexoses by adsorption on sulfonated polystyrene divinylbenzene crosslinked ion exchange resins exchanged with calcium-ammonium cationic and desorbing the adsorbate with water. But both the two inventions did not relate to recover the xylose or xylitol.

Chromatographic separation effect of the monosaccharides and the furfuryl alcohol by ion exchange resin or zeolite (water as the eluent) depends on their hydrophobic/hydrophilic property. The monosaccharides having same carbon atoms (such as xylose and arabinose), or furfuryl alcohol having same carbon atoms (such as xylitol and arabitol), they are isomeride to each other and has similar chemical property, thus the same cation exchange resin has little different absorption property between them. So whether separate the xylose or arabinose directly, or separate the corresponding alcohols inverted by the monosaccharides, there are difficult such as lower separate efficiency and lower production and purity.

To improve the recovering efficiency, fermentation has been adopted to purify the product by many researchers, i.e. remove the needless saccharides by microbes to relatively increase the content of target saccharides, thus to improve the efficiency of the next separating process.

Nyun etc. (Nyun Ho Park, Shigeki Yoshida, Akira Takakashi, et al. A new method for the preparation of crystalline L-arabinose from arabinoxylan by enzymatic hydrolysis and selective fermentation with yeast. Biotechnology Letters 2001, 23:411-416) use a crude enzyme from Penicillium funiculosum culture to hydrolyze the corn fiber rich in Arabinoxylan (containing 28.1% arabinose and 32.8% xylose), as a result, 21.3% (w/w) arabinose and 18.7% (w/w) are hydrolyzed. In addition, the hydrolysates contains the other monosaccharides and oligosaccharide. the hydrolysates then treated by Williopsis saturnus which can consume xylose and can not consume arabinose in 96 h, as a result, 95% arabinose is remained in hydrolysates, and only 0.002% xylose (relatice to original Arabinoxylan is existed. The fermentation method selectively remove the xylose overcome the difficult of separating the arabinose and the xylose, but the xylose is waste.

LI Dao-yi etc. (Preparation of Crystalline L-arabinose from Corn is Seed-coat Acid Hydrolysis Fermented by Specific Yeast, Food Science, 2007, 28 (4): 125-127), the method of L-arabinose crystalline preparation was carried out in the report. After corn hull being hydrolyzed by the diluted sulfuric acid, the hydrolysis solution was found mainly consisting of arabinose, xylose and glucose. Yeast WYSI5-3 was aerobically cultured in the hydrolyzed solution followed by neutralizing with calcium carbonate to remove xylose and glucose. The result solution was decolorized, desalted and concentrated, L-arabinose is obtained as crystalline state from aqueous ethanol, and the yield is 9.6% based on corn seed-coat dry mass. In this method of getting arabinose, the xylose is wasted and the period is too long (7 days). In addition, because the used yeast can not assimilate the galactose in the hydrolysates, the hydrolysates in the arabinose solution directly affect the crystal rate.

CN application number 200510040433.0 (Process for extracting xylose and xylitol from a xylose mother liquor or a xylose digest) relates to a process for extracting xylose and xylitol from a xylose mother liquor or a xylose digest, which comprises, using xylose hydrolysate or xylose mother liquid as the raw material, removing glucose through saccharomyces cerevisiae fermentation, then imitating moving bed, using water as eluent, separating xylose from foreign matter such as arabinose, thus obtaining component containing rich xylose. However, in the embodiments of the invention, the xylose can not be completedly separated from the foreign is matter such as arabinose, or the separate the xylitol from arabitol. In addition, the invention did not relate how to selectively convert the xylose into xylitol and then separate the xylitol from the other saccharides.

Because the ion chromatography using water as eluent has the default, the other methods are attempted to overcome the problem.

US20060100423 (Process for the preparation and separation of arabinose and xylose from a mixture of saccharides) relates to a process for the preparation and separation of the pentoses, xylose and arabinose from mixtures of saccharides by forming acetals. D-xylose is a precursor to xylitol, a sweetener, and L-arabinose is a precursor to the drug intermediate (R)-3,4-dihydroxybutyric acid, carnitine and agrichemicals. But using much organic solvent is obviously having disadvantages in production.

FENG Ya-qing etc. (Extraction and Purification of L-Arabinose from Arabic Gum, Fine Chemicals, 2003, 20(5): 288-290), arabic gum was hydrolyzed with sulfuric acid solution to give the mixed solution containing L-arabinose, which was concentrated and extracted with 90% alcohol and precipitated by industrial acetic acid to give the crude L-arabinose. After chromatographic separation with double column, the is yield was more than 50% and the purity was 99%. But the research is not use the hemicellulose from cell wall of plant as the material, and chromatographic separation method with organic solvent as the mobile phase has potential safety hazard in mass industrial production.

In summary, we can see clearly that the chemical process of industrially produce xylitol or directly recover arabinose from hemicellulose hydrolysates in prior art can not overcome the problem of being very difficult to separate xylose and the arabinose. In addition, because the no-selectivity property of the chemical reduction, the chemical technology of the prior art can not directly use hemicellulose hydrosylates to produce xylitol and arabinose at same time.

Microbes can catalyze the xylose into xylitol. The hemicellulose hydrolysates being directly fermented by the microbes to produce xylitol has the advantages such as energy saving, need not the purification process necessary to chemical technology, thus it has attracted great attention. However, the efficiency of producing xylitol by fermentation is too lower, and most researches only focus on the preparation of the hemicellulose hydrolysates or optimizing of the fermentation of the xylitol. Little research is focus on obtaining xylitol from the xylose fermentation liquor, and no research is relates to combine the fermentation of the xylose with the preparing of arabinose.

The inventor of the present invention (CAI Ai-hua; ZHANG Hou-rui; HE Cheng-xin etc. Xylitol Purification from the Fermentative Broth of Sugar Cane Bagasse Hemicellulose Hydrolysate, Food Science, 2006, 27(7): 136-139) had found that after ultrafiltration, the filter liquor of the fermentative broth of sugar cane bagasse hemicellulose hydrolysate is obtained, After desalination by ion-exchange column process, followed by decoloration with active carbon, the purified xylitol syrup was concentrated up to soluble solids 80%. Then, the xylitol product could be crystallized out with purity more than 98.5%. In this research the inventor also observed that in the crystalline mother liquor, if the concentration of the arabinose is higher than 45.0% of xylitol, or the concentration of xylose is higher than 12.6% of xylitol, this two will crystallize out at same time as the xylitol crystallizes. It is difficult to efficiently purify the xylitol from such mother liquor only by the crystallization process alone if the concentration of the xylose or arabinose is out of the range. This characteristic of the technology is that directly concentrate and crystallized the purified fermentation broth to obtain the crystal production of xylitol, and remain the crystallized mother liquor which is hard to be crystallized and contains xylitol, arabinose, and xylose. The inventor did not think to combine the fermentation of the xylose with the preparing of arabinose then.

Ding Xinghong etc. (Effects of Several Key Factors on Xylitol Separation and Purification in Fermented Hemicellulose Hydrolyzates, Journal of Chinese Institute of Food Science and Technology, 2006, 6(6):87-90), in order to optimize the xylitol crystallization technology for the fermented hemicellulose hydrolyzates, two sets of crystallization tests were performed on both xylitol solutions and fermented hemicellulose hydrolyzates. The influences of xylitol is concentration, remained sugar(arabinose), temperature and crystal seeds on the kinetics of xylitol crystallization were investigated. The optimum initial xylitol concentration of crystallization media was about 750 g/L, and the optimum crystallization temperature was −4° C. By adding 1% xylitol crystal seeds, crystallization time was shortened, and improved crystallization rate was obtained. The arabinose present could increase the xylitol crystallization rate, however, the xylitol crystal purity deteriorated when the arabinose concentration exceeded 120 g/L. In this research, the technology is also to concentrate the purified fermented hemicellulose hydrolysates and then crystallize to obtain xylitol. Although it mentioned that the arabinose present could increase the xylitol crystallization rate, but it not mention how to separate the xylitol from the arabinose.

YING Guo-Qing etc. (Separation and Purification of Xylitol Produced by Biotransformation, Chinese Journal of Pharmaceuticals, 2002, 33(3): 117-123) shown that as the volume ratio of the fermentation solution contained 0.20% xylose to 2.0 mol/L NaOH was 1:29, the residual xylose was hydrolyzed to produce corresponding acid and salt after reflux for 2 h. Then the acid and salt were removed by ion-exchange resins. The xylitol was crystallized. Obviously, this technology did not prepare to recover the other saccharide in the fermentation solution.

In chromatographic separation of xylose, arabinose and xylitol by calcium type cation exchangers, we found that the chromatographic peak of xylose and that of arabinose are mostly overlapped, but their peaks are hardly overlap with the peak of the xylitol (FIG. 3). Obviously, the chromatographic separation efficiency between xylitol-arabinose (saccharide-alcohol) is much higher than that between xylose-arabinose (saccharide-saccharide). If we can selectively convert the xylose in the hemicellulose hydrolysates into xylitol, and let the arabinose no to react, then the separation between xylose and the arabinose will be changed to separation between the xylitol and the arabinose, then the difficult between the xylose and the arabinose is solved.

Some yeast strains have the property of capable of consuming glucose, converting the xylose into xylitol and can not utilize arabinose. If the hemicellulose hydrolysates mostly consisting xylose-arabinose-glucose is fermented by these yeast strains, then liquor mostly consisting xylitol-arabinose will be fermented. Thus if the hemicellulose hydrolysates is fermented by these yeast, both the bio-transformation (xylose converted to xylitol) and the bio-purification (the glucose is consumed, thus the purity of the xylitol and arabinose is relatively improved) are achieved, and the efficiency of the chromatographic separation of the next production will be improved. And one chromatographic separation of the fermented liquor can purify both the xylitol and arabinose at the same time, thus can avoid strictly selecting the material in the production of the xylitol or arabinose, and can improve the efficiency of material usage and decrease the cost.

### SUMMARY OF THE INVENTION

The primary object of the present invention is to obviate the disadvantages that in prior art the detoxification process of the hemicellulose hydrolysates is complicated and high cost.

The present invention comprising: after enrichment, separation and screen of the sludge samples from the soil around the paper mill, xylose mill and furfural mill, two separations: S-7 and Lj-3 which can improve the fermentation property of the cellulose hydrolysates are obtained. The separation S-7 is identified to belong to Issatchenkia orientalis, and can not utilize the xylose; the separation lj-3 is identified to belong to Issatchenkia occidentalis; and utilize little xylose. The invention providing a detoxification method for the hemicellulose hydrolysates by the new strains: mix the new strains and the strain which can ferment the xylose into xylitol, thus the hemicellulose hydrolysates can be detoxified and produce xylitol at same time; mix the new strains and the strain which can ferment the xylose into the alcohol, thus the hemicellulose hydrolysates can be detoxified and produce alcohol at same time. The strain conservation information of the two strains can be seen in the two strain conservation information notices.

The isolation, screen, and identification of the two strains S-7 (CCTCC NO:M206098) and Lj-3 (CCTCC NO:M206097) of the present invention are as follows:

Hemicellulose hydrolysate of sugar bagasse is used as the basic component as the isolation medium, after properly vacuum, the PH is adjusted to 5-6 by alkali. If the medium is to be plating medium, add 20 g/L agar as forming agent.

The isolation samples are the soil and silt collected from the surroundings polluted by the waste water from the pulp mill. Each 250 ml flask is added in 25 ml medium and then is added 1 g sample, under conditions of 30° C., 200 rpm shaking cultured for 72 h. The culture solution with growing microbes isolated in streak plate, select the fast grow strains and they are saved in slant culture medium.

Slant culture is transferred to the hemicellulose diluted acid hydrolysates and shaking cultured for 24 h, centrifugation to remove the microbes body and inoculated with yeast strain which can convert the xylose and then ferment, select the strains which can effectively improve the fermentation property of hemicellulose diluted acid hydrolysates. Thus by more than ten batches of sample isolation, two isolations which have the most strong ability of improving the fermentation property of hemicellulose diluted acid hydrolysates are obtained, and their serial number are S-7 and Lj-3 respectively.

The detecting insult by HPLC (High Performance Liquid Chromatography) shows that both the S-7 and Lj-3 have high degrading activities to the acetic acid, furfural and phenols, the representative toxic in the hemicellulose diluted acid hydrolysates. Herein the S-7 does not consume xylose and Lj-3 consume little xylose.

The two strains isolated by above method are saved in 4° C. or freeze drying.

According to the methods provided by [symbol] yeast strains identifying manuals [symbol] (Ocean University of China publishing house), the cell character, colony character and physiological and biochemical property of the isolations S-7 and Lj-3 are identified (table 1 and table 2). The S-7 has the same physiological and biochemical property to the Issatchenkia orientalis described in the [symbol] yeast strains identifying manuals [symbol] ; the Lj-3 has the same physiological and biochemical property to the Issatchenkia occidentalis described in the [symbol] yeast strains identifying manuals

[symbol]

Amplify the D1/D2 region sequences of the 26S rDNA of S-7 and Lj-3 by PCR with primer 5′-GCATATCAAAAGCGGAGGAAAAG-3′ and 5′-GGTCCGTGTTTCAAGACGG-3′ (referring to Kurtzman C P, Four new Candida species from geographically diverse locations. Antonie van Leeuwenhoek, 2001, 79: 353-361), identify the sequences of the amplified products (table 3 and table 4), and the sequences are Nucleotide-nucleotide BLAST in GenBank. The result shows that, the homology between the D1/D2 region sequence of 26S rDNA of S-7 and that of the Issatchenkia orientalis is 100%, the homology between the D1/D2 region sequence of 26S rDNA of Lj-3 and that of the Issatchenkia occidentalis is 100%.

According to the cell character, colony character and physiological and biochemical property, S-7 can be divided to Issatchenkia orientalis and Lj-3 can be divided into Issatchenkia occidentalis in classification.

the conservation number of Issatchenkia orientalis Lj-3 in China Centter For Type Culture Collection is CCTCC NO:M206098, and the D1/D2 region sequence of 26S rDNA of S-7 in GenBank is number EF030708

http://www.ncbi.nlm.nih.gov/entrez/viewer.fcgi?db=nucleotide&val=116834296

the conservation number of Issatchenkia orientalis Lj-3 in China Center For Type Culture Collection is CCTCC NO:M206097, and the D1/D2 region sequence of the 26S rDNA of S-7 in GenBank is number EF030710

http://www.ncbi.nlm.nih.gov/entrez/viewer.fcgi?db=nucleotide&val=116834298.

TABLE 1
the Cell characteristics and colonial morphology of isolation
S-7, Lj-3

| | colonial morphology | Cell characteristics |
|---|---|---|
| S-7 | Drying surface, edge | multilateral budding, average cell |
| | radiately. | length: 10~20um. |
| Lj-3 | glossy, mucoid surface, | multilateral budding, average cell |
| | edge neatly. | length: 5~15um. |

TABLE 2
physiological and biochemical property of S-7 and Lj-3(+,
utilize; −, can not utilize)

| C/N | S-7 | Lj-3 | C/N | S-7 | Lj-3 |
|---|---|---|---|---|---|
| glucose | + | + | D-xylose | − | little |
| D-galactose | − | − | L-sorbose | − | − |
| maltose | − | − | D-ribose | − | − |
| α-methyl-D- | − | − | dulcitol | − | − |
| glucopyranoside | | | | | |
| sucrose | − | − | D-sorbic | − | − |
| | | | alcohol | | |
| fucose | − | − | Succinic | + | + |
| | | | acid | | |
| melibiose | − | − | glycerin | − | − |
| lactose | − | − | inositol | − | − |
| cellobiose | − | − | ethanol | + | + |
| melezitose | − | − | D-fructose | − | − |
| raffinose | − | − | DL-malic | − | − |
| | | | acid | | |
| inulin | − | − | urea | + | + |
| Soluable starch | − | − | Lactic | + | + |
| | | | acid | | |
| Citric acid | + | − | L-rhamnose | − | − |
| carbinol | − | − | | | |

Additional test:

| | | | | | |
|---|---|---|---|---|---|
| 50% glucose | little | − | 13~14% | − | − |
| | | | Nacl | | |
| 60% glucose | − | − | | | |

Sequence of the 26S rDNA D1/D2 region of the Issatchenkia orientalis S-7 (CCTCC NO:M206098) (GenBank number EF030708)

| | |
|---|---|
| 1 | taagcggagg aaaagaaacc aacagggatt gcctcagtag cggcgagtga agcggcaaga |
| 61 | gctcagattt gaaatcgtgc tttgcggcac gagttgtaga ttgcaggttg gagtctgtgt |
| 121 | ggaaggcggt gtccaagtcc cttggaacag ggcgcccagg agggtgagag ccccgtggga |
| 181 | tgccggcgga agcagtgagg cccttctgac gagtcgagtt gtttgggaat gcagctccaa |
| 241 | gcgggtggta aattccatct aaggctaaat actggcgaga gaccgatagc gaacaagtac |
| 301 | tgtgaaggaa agatgaaaag cactttgaaa agagagtgaa acagcacgtg aaattgttga |
| 361 | aagggaaggg tattgcgccc gacatgggga ttgcgcaccg ctgcctctcg tgggcggcgc |
| 421 | tctgggcttt ccctgggcca gcatcggttc ttgctgcagg agaaggggtt ctggaacgtg |
| 481 | gctcttcgga gtgttatagc cagggccaga tgctgcgtgc ggggaccgag gactgcggcc |
| 541 | gtgtaggtca cggatgctgg cagaacggcg caacaccgcc cgtcttgaaa cacgga |

Sequence of the 26S rDNA D1/D2 region of the Issatchenkia occidentalis) Lj-3 (CCTCC NO:M206097) (GenBank number EF030710)

| | |
|---|---|
| 1 | tatcaataag cggaggaaaa gaaaccaaca gggattgcct cagtagcggc gagtgaagcg |
| 61 | gcaaaagctc agatttgaaa tcgtgtttcg gcacgagttg tagattgcag gttggagtct |
| 121 | ttgtggaagc gtgtgtctaa gtcccttgga acagggtgcc attgagggtg agagccccgt |
| 181 | gagacgcgtg cggaagctgt aaggcccttc tgacgagtcg agttgtttgg gaatgcagct |
| 241 | ctaagtgggt ggtaaattcc atctaaggct aaatattggc gagagaccga tagcgaacaa |
| 301 | gtactgtgaa ggaaagatga aaagcacttt gaaaagagag tgaaacagca cgtgaaattg |
| 361 | ttgaaaggga agggtattgg gctcgacatg ggatttgcgc accgctgctc cttgtgggcg |
| 421 | gcgctctgtg cttttcctgg gccagcatcg gtttttgccg caggagaagg cgtgctggaa |
| 481 | tgtggctctt cggagtgtta tagccagtgc gagatgctgc gtgcggggac cgaggactgc |
| 541 | gacatctgtc tcggatgctg gcacaacggc gcaataccgc ccgtcttgta a |

The hemicellulose hydrolysate of the present invention is prepared by:

Crop straw such as cane sugar bagasse, corncob or straw etc. are crushed into proper granularity, and then add 0.3˜3% (w/w) diluted sulfuric acid or hydrochloric acid according to solid-to-liquid ratio 1:6˜7, and mix up, heated to 100-130° C. in a pressure anti-acid vessel and maintained for 0.5˜3 h. After hydrolyzing, remove the residue, then the filtrate is the hemicellulose hydrolysate. The PH of the hemicellulose hydrolysate is adjusted to 3˜8 by solid CaCO3 or Ca(OH)2, then detoxified directly or after concentration. Usually, in higher acid condition, it can be lower hydrolyzing temperature or shorter hydrolyzing time, in lower acid condition it must be higher hydrolyzing temperature or longer hydrolyzing time

The microbes used for detoxification of the hemicellulose hydrolysates, xylitol fermentation and ethanol fermentation of the present invention are cultured by:

Culture of the seed liquor of the detoxification strains: slant culture of the CCTCC N0:M206098 or CCTCC N0:M206097 is added into liquid seed medium, the liquid is added to 20% volume of the flask, under 27-30° C., 200 rmp shaking conditions cultured for 12-18 h, then the seed liquor is acquired. The seed medium consisted by: Glucose 50 g/L, MgSO₄.7H₂O 2 g/L, K₂HPO₄ 4 g/L, KH₂PO₄ 6 g/L, yeast extract 5 g/L.

Culture of the seed liquor for ferment the xylose into xylitol: the strain is Candida tropicalis CCTCC N0:M205067 (referring to china patent application number 200510037580.2, isolation of the Candida tropicalis and its application), the medium comprising 20 g/L glucose and 20 g/L xylose, and the other components and culture condition is the same as the culture of the seed liquor of the detoxification strains.

The detoxification of the hemicellulose hydrolysates is processed by:

The total sugar content of the hemicellulose hydrolysates is between 4˜20% (w/w), wherein the reducible saccharides are more than 90% of the total sugar, wherein the xylose is the main component. Adjust the PH into 3-7 by lye (Ca(OH)₂, ammonia etc.), then inoculate the cultured seed liquor of CCTCC NO:M206098 or CCTCC NO:M206097 by 10% (v/v) inoculation, under 25˜35° C., aerobically cultured and detoxified for 5-20 h, herein most toxic components for the microbes will be degraded. Collect the strains after centrifugation and they are reused for the detoxification of the next batch of fresh hemicellulose hydrolysates, the supernatant can be used for producing xylitol or ethanol directly or after concentration.

The process of produce xylitol by hemicellulose hydrolysates after detoxification of the present invention is by:

Hemicellulose hydrolysates after detoxified by CCTCC NO:M206098, or CCTCC NO:M206097 is concentrated into about 150 g/L xylose under vacuum, and PH is adjusted to 6 by ammonia, add 5 g/L yeast extract, then the hemicellulose hydrolysate medium for fermenting xylitol is obtained. The xylitol fermentation is processed by flask, filling 10% volume of the flask, and according to 5% (v/v) inoculation to inoculate the seed liquor of Candida tropicalis CCTCC NO:M205067, under 200 rpm and 33° C. conditions to ferment until the xylose is all consumed. Collect the strain cells after centrifugation and they are re-used for xylitol fermenting of the next batch fresh hemicellulose hydrolysate, the supernatant is used for produce xylitol.

The process of producing xylitol with detoxification in the same time of the hemicellulose hydrolysate of the present invention is by:

Hemicellulose hydrolysates is concentrated into about 100-150 g/L xylose under vacuum, and PH is adjusted to 3 to 7 by lye (Ca(OH)2, ammonia etc.), then centrifugated or filtrated to remove residues, and add 5 g/L yeast extract, the hemicellulose hydrolysate medium for fermenting xylitol is obtained, sub-pack the medium in flasks. Seed liquor of cultured CCTCC NO:M206098 or cultured CCTCC NO:M206097 is mixed with same volume seed liquor of CCTCC NO:M205067, and according to 5% (v/v) inoculation to inoculate to the hemicellulose hydrolysates under shaking condition fermenting until the xylose is all consumed. Collect the yeast cell after centrifugation and they are re-used for next batch fresh hemicellulose hydrolysates until the yeast cell can not be used.

Hemicellulose hydrolysate detoxified in advance is fermented to produce xylitol and arabinose, the steps comprising:

S1. Hemicellulose hydrolysates detoxified by CCTCC NO:M206098 is concentrated into about 100-150 g/L xylose under vacuum condition, and PH is adjusted to 6 by ammonia, then add 5 g/L yeast extract, the hemicellulose hydrolysates medium for fermenting xylitol is obtained.

S2. The xylitol fermentation is processed by flask, filling volume is 10% volume of flask, according to 5% (v/v) inoculation the seed liquor of cultured CCTCC NO:M205067 is inoculated into the hemicellulose hydrolysate, under 200 rpm and 33° C. conditions to ferment until the xylose is all consumed.

S3. collect the strains after centrifugation and they are re-used for fermenting xylitol of the next batch of fresh hemicellulose hydrolysates, the supernatant is used for producing xylitol.

S4. remove the cell of the fermented xylitol liquor, purified by ion exchange, then calcium type cationic resin chromatographic separation to obtain the pure xylitol diffluence, the xylitol diffluence is concentrated and crystallized to obtain crystalline xylitol.

S5. after diffluent of the xylitol, the other liquor is arabinose diffluent and is purified by ammonium type cationic resin chromatographic separation to improve the purity of the arabinose, then concentrated and crystallized to obtain crystalline arabinose.

The process of producing xylitol and arabinose in the same time with detoxification of the hemicellulose hydrolysates of the present invention is by:

S1. Hemicellulose hydrolysates is concentrated into about 100-150 g/L xylose under vacuum, and PH is adjusted to 3 to 7 by lye (Ca(OH)₂, ammonia etc.), then add 5 g/L yeast extract, the hemicellulose hydrolysates medium for fermenting xylitol is obtained.

S2. seed liquor of cultured CCTCC NO:M206098 is mixed with same volume seed liquor of CCTCC NO:M205067, and they are inoculated to the hemicellulose hydrolysate according to 5% (v/v) inoculation, under shaking condition, ferment until the xylose is all consumed.

S3. collect the yeast cell after centrifugation and they are re-used is for next batch fresh hemicellulose hydrolysate until the yeast cells can not be used.

S4. after being removed the cell, the fermented xylitol liquor is purified by ion exchange, then calcium type cationic resin chromatographic separation is used to obtain the pure xylitol diffluence, the xylitol diffluence is concentrated and crystallized to obtain crystalline xylitol.

S5. after the xylitol diffluent, the other liquor is arabinose diffluent and is purified by ammonium type cationic resin chromatography to improve the purity of the arabinose, then concentrated and crystallized to obtain crystalline arabinose.

The degrading activity to the representative toxic components and main phenols in the hemicellulose hydrolysates of the stains of the present invention is detected by HPLC.

Acetic acid, furfural and guaiacol are selected as representation of different type inhibitors in hemicellulose hydrolysates, and they are added into common yeast medium and inoculated in CCTCC NO:M206097 or CCTCC NO:M206098 to ferment. Because each of the three compounds has strong ultraviolet absorption in 198 nm, in the following chromatogram conditions: Waters486 High Performance Liquid Chromatograph, ZORBAX XDB-C18 chromatographic column, mobile phase is carbinol:phosphoric acid (0.2%, w/w)=75:25 (v/v), compare the content difference of the three compounds before and after fermentation, the results show that the two strains have degrading activity to all of the three compounds (FIG. 1).

Because phenols have strong ultraviolet absorption in 270 nm, the bagasse hemicellulose hydrolysate in operation 1 is compared with the bagasse hemicellulose hydrolysate detoxified by CCTCC NO:M206098 or CCTCC NO:M206097 by HPLC in 270 nm (FIG. 2), and analyse the information of the characteristic peaks in the detecting conditions (table 4), it shows that after detoxification, the furfural and many phenols in the is hemicellulose hydrolysates are degraded.

The detoxification activity of the stains of the present invention to the hemicellulose hydrolysates is detected by bio-fermentation comparation.

Compared the fermentation results of CCTCC NO:M205067 fermenting the hemicellulose hydrolysate before or after being detoxified by CCTCC NO:M206097 or CCTCC NO:M206098, and detect the fermentation product—xylitol; As a result, the hemicellulose hydrolysate detoxified by the strains and methods of the present invention greatly improved the yield and the product producing rate for fermenting xylitol. Namely, the strains and methods of the present invention can effectively improve the fermentation property of the hemicellulose hydrolysates.

The advantages of the present invention are as follows:

1. the strains CCTCC NO 206097 and CCTCC NO 206098 of the present invention have degrading activity to the three representative toxic compounds in the hemicellulose hydrolysates: the organic acid (as a representation: acetic acid), degrading products of glucide (as a representation: furfural), and phenols (as a representation: guaiacol), and the strains have obvious bio-detoxification activity to the hemicellulose hydrolysates.

2. the strains CCTCC NO:M 206097 and CCTCC NO:M 206098 of the present invention are used to degrade many toxic compounds in the hemicellulose hydrolysates, this decrease the impurity in the matrix. Compared to the physical and chemical detoxification in prior art, the present invention obviously has advantages such as simple process, lower cost and friend to circumstance.

3. in the two strains of the present invention, CCTCC NO:M 206097 can not consume xylose, CCTCC NO:M 206098 consumes little xylose, the two is strains of the present invention are used to detoxify the hemicellulose hydrolysates and this will not decrease the xylose in the hydrolysates. By this character, they are added into a fermentation system with the microbes which can convert xylose into xylitol or convert xylose into ethanol, thus can realize detoxification coupling with xylitol fermenting or ethanol fermenting of the hemicellulose hydrolysates, this greatly simplified the process of producing xylitol or ethanol by hemicellulose hydrolysates.

In particular, the present invention provided a new method of combining the xylitol fermenting of the hemicellulose hydrolysates with producing arabinose from hemicellulose hydrolysates, this effectively solve the problem that the lower recovering efficiency and lower yield by the similar chemical character between the xylose and arabinose in chemical xylose producing process and recovering arabinose from hemicellulose hydrolysates, or the potential safety hazard by using organic solvent. Compared with the process of recovering xylitol from hemicellulose hydrolysates, the present invention can produce xylitol in addition. Compared with the arabinose producing process in prior art, the present invention can produce xylitol in addition. Obviously, the present invention can increase the use ratio of resource, and lower the producing cost of xylitol and arabinose.

The process of the present invention comprising: raw materials rich in xylan are hydrolyzed by acid or enzyme to obtain hydrolysate rich in xylose and arabinose with impurity saccharides such as glucose, mannose and galactose. After being detoxified to remove the inhibitors to the microbes, the hydrolysate is inoculated into yeast stains which can consume glucose, and convert xylose into xylitol and can not consume arabinose. When the concentration of the xylose in the fermentation is is lower to certain content, the fermentation is stopped and hydrolysates with main components of xylitol and arabinose is obtained. Hydrolysates is processed by: remove the cell, purified by ion exchange, and decolored, concentrated, then calcium type cationic resin chromatographic separation with water as eluent, the pure xylitol diffluence with purity up than 99% and diffluence with arabinose-sugars are obtained, the xylitol diffluence is concentrated and crystallized to obtain crystalline xylitol. Arabinose-sugars diffluence is purified by ammonium type cationic resin chromatography to improve the purity of the arabinose, then concentrated and crystallized to obtain crystalline arabinose.

Although the chromatographic separation can be finished in stationary phase columns filled with cation resin, but industrial simulated moving bed can improve the separating efficiency.

FIG. 4 shows the total process of the invention.

The hemicellulose hydrolysates of the present invention is obtained by: material rich in hemicellulose such as sugar cane bagasse, corn fiber or corn cob etc. are added with 0.5˜2.5% (w/w) diluted sulfuric acid or hydrochloric acid to covering the material, heated to 100˜140° C. and maintained for 0.5˜2.5 h. After hydrolyzing, remove the residue, the PH of the hemicellulose hydrolysates is adjusted to 3˜4 by solid CaCO3 or Ca(OH)₂, and remove the residue, then the supernatant is treated by active carbon according to 1-3% weight of the material. After carbon being removed, the liquor is pass through the cation resin and anion resin in turn, then is concentrated, thus the hemicellulose hydrolysates for fermentation is obtained.

By following steps the xylose of hemicellulose hydrolysates is selectively bio-catalyzed in ferment pot by yeast cell while the arabinose is not bio-catalyzed.

The microbes of the present invention is Candida tropicalis which can consume the hexose such as glucose, mannose and galactose in the hemicellulose hydrolysates and will not produce corresponding sugar alcohol. According to pentose, the Candida tropicalis can convert the xylose into xylitol while can not consume the arabinose. The strains Candida tropicalis CCTCC M 205067 of the present invention is isolated by the inventor, and is conserved in CCTCC (china application number 200510037580.2, publication number CN1982460)

The seed medium consisted by: xylose 20 g/L, Glucose 30 g/L, yeast extract 10 g/L, KH₂PO₄, 5 g/L, NH₄H₂PO₄, 3 g/L, MgSO₄.7H₂O, 0.1 g/L, pH 5-6, liquor is filled in 10-20% of the volume of the flask, sterilized for 15 minutes in 115° C. slant culture of the yeast is inoculated to the cooled seed medium, 28-35° C., shaking cultured for 10-12 h, then seed culture liquor is inoculated into fresh medium according to 5-10% (v/v) inoculation, shaking culture to acquire enough liquor seed.

The hemicellulose hydrolysate is concentrated to be the total saccharides being about 200-250 g/L, then is filled into a ferment pot, 50-150 g hot water extract of the rice bran or wheat bran is added to per litre hemicellulose hydrolysate to meet the growth need of nutrition for the yeast. Inoculated the yeast seed according to 5-10% (v/v) inoculation, 33° C. aerobically cultured until the xylose is all consumed.

After each batch is finished, the yeast cells are separated from the ferment liquor by centrifuge or filtration, then the ferment liquor is used to produce xylitol and arabinose, the collected cells are transported into fresh hydrolysates medium for next ferment. The fresh medium has the same xylose concentration, same ingredients, and same filling quantity in the ferment pot. Repeat such steps until the cells can not be used.

In the present invention, the xylitol and the arabinose in the ferment liquor are separated by:

ferment liquor which has been removed the cells is firstly ultrafiltrated to remove proteins, amylose and part of pigment, then decolored by activated carbon and desalted by ion exchange resin, thus transparent xylitol ferment liquor is obtained. In reduced pressure the xylitol is concentrated to 40-60% (w/w) to first chromatographic separation.

Adding sample on the top of chromatographic column filled with calcium type cation resin, and open the bottom of the column, when the sample is fully in the column, eluted by 30-90° C. pure water. The first chromatographic peak flow out the bottom is mixture of saccharides mostly consisted by arabinose, the second chromatographic peak is xylitol with little impurity, and the purity is beyond 99%. And the two chromatographic peaks have little overlap portion.

The xylitol diffluence of the chromatographam is concentrated to 90% (w/w) in reduced pressure condition, then cooled and crystallized to obtain pure crystalline xylitol.

The saccharides diffluence of the first chromatographic separation is concentrated to total saccharides 40-60% (w/w), adding sample on the is top of chromatogram column filled with ammonium type cation resin, and pure water elute for second chromatographic separation. The first chromatographic peak flow out the bottom is mixture of saccharides, the second chromatographic peak is arabinose, collect the second chromatographic peak, then concentrated in reduced pressure condition to total saccharides 60-80% (w/w), then cooled and crystallized to obtain pure arabinose crystals.

If same cation resin is used as the fillings of the simulated moving bed, the xylitol and the arabinose can be more effectively separated than columns, this will greatly improved the producing efficiency of per weigh resin and save water.

Compared with the prior art, the present invention has the following advantages according to producing the arabinose.

Firstly, the hemicellulose hydrolysates is fermented by special yeast strains of the present invention to consume most saccharides by cell metabolism, and the xylose is effectively converted into xylitol and the arabinose is remained no-act. Thus one process can get two products, this is not researched by any prior art.

Secondly, by the selective bio-catalysis, the saccharide-saccharide separating in prior art between the xylose-arabinose is transformed to be the saccharide-alcohol separating of xylitol-arabinose, after chromatographic separation, 99% xylitol can be get from the purified liquor, and the yield is close to 100%. This two data are not reported in any researches in prior art.

Thirdly, the present invention obtain high purity xylitol liquor directly after first chromatographic separation, the next crystallization step is a physical forming step of the product and is not the method of purification, so there are not any un-usable xylitol crystallization mother liquor in present invention, this is not realized in prior art.

Fourthly, because after the first chromatographic separation the xylitol is totally recovered, and the second chromatographic separation is to separate the arabinose from the mixed saccharides, this is more easier than the separation between the arabinose-xylose. after second chromatographic separation, the purity of arabinose is raise to more than 85%, this is not realized in prior art.

Fifthly, the present invention broaden the material resource for producing xylitol or arabinose. Whether produce xylose or arabinose by hemicellulose hydrolysates, the object saccharide content in the material must high, or else the object saccharide cannot be crystallized out. For example, in prior art in produce arabinose must choose material rich in araban, and strictly control the acid dosage to reduce the degrading of the main chain of the xylan so as to decrease the xylose content in the hydrolysates, thus the arabinose crystallizations can be obtained. Similarly, in prior art in xylose producing the material rich in arabinose can not be used. But in present invention, the xylose can be converted into xylitol and separated from arabinose in chromatographic separation in high content arabinose conditions. So, whether what the ratio between the arabinose and the xylose in the material, the present invention can produce high purity xylitol and arabinose at same time.

Sixthly, compared with arabinose producing technology in prior art, the present invention more effectively utilize the xylose of the hydrolysates and convert it into more valuable xylitol and then recovered. While in prior art, they must try to reduce the hydrolyzation of the xylose in material, the hydrolyzed xylose is to be consumed by yeast cell or existed in the crystallizations mother liquor and is not effectively utilized.

Seventhly, compared with xylitol producing technology in prior art, the present invention more effectively utilize the xylose and the arabinose of the hydrolysates. In prior art, the xylose must be firstly crystallized and purified from the hydrolysates, then hydrogenate to produce xylitol, so there are much xylose and arabinose mixed in the mother liquor and cannot be utilized. The process of recovering xylitol from ferment liquor in prior also has much arabinose in crystallization mother liquor. In present invention, the xylitol is completely recovered and the arabinose is effectively recovered.

### BRIEF DESCRIPTION OF THE DRAWINGS

FIG. 1 shows the change of HPLC of the inhibitor degraded by stains. Parameter: (A) inhibitor, (B) medium, (C) medium added with inhibitor, (D) medium containing inhibitor fermented by CCTCC NO:M206097, (E) medium containing inhibitor fermented by CCTCC NO:M206098. Peaks and compounds: 1 acetic acid, 2 furfural, 3, guaiacol, 4, 5 components of medium, 6, degraded product of furfural, 7 degraded product of guaiacol.

FIG. 2 is a HPLC fingerprint before and after the bio detoxification of the sugar cane bagasse hemicellulose hydrolysates (270 nm). Parameter: A furfural sample; B sugar cane bagasse hemicellulose hydrolysates; C, sugar cane bagasse hemicellulose hydrolysates detoxified by CCTCC NO:M206098; D, sugar cane bagasse hemicellulose hydrolysates detoxified by CCTCC NO:M206097.

FIG. 3 is a separating curve of xylose, arabinose and xylitol by calcium type cation resin chromatogram.

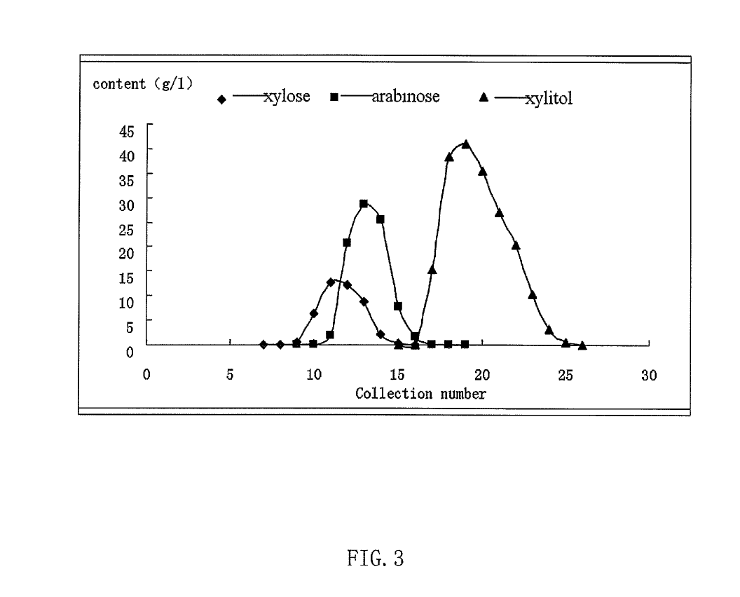

FIG. 4 is a chart of producing xylitol and arabinose by hemicellulose hydrolysates.

FIG. 5 is a HPLC chromatogram of the hemicellulose hydrolysates of the corn fiber (1, glucose, 2, xylose, 3, arabinose).

FIG. 6 is a HPLC chromatogram of the fermented liquor of the hemicellulose hydrolysates of the corn fiber (1, xylose, 2, arabinose, 3, xylitol).

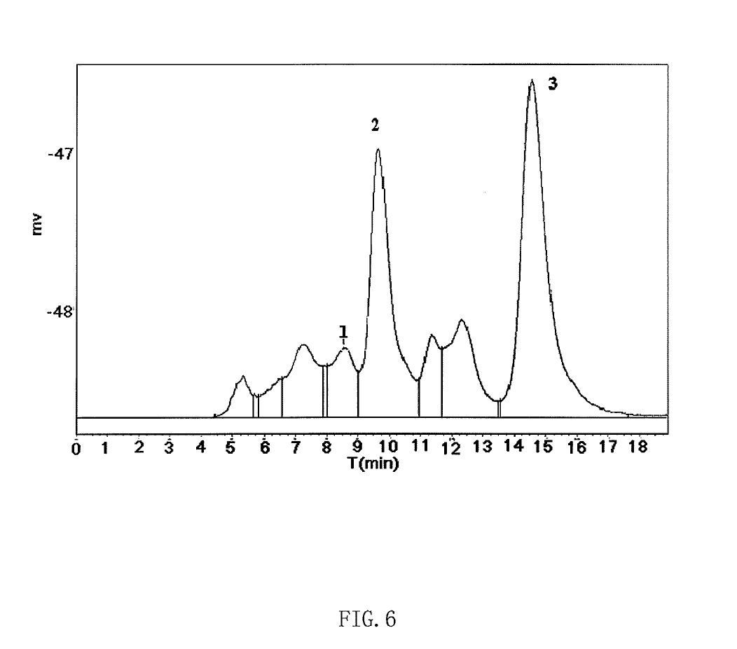

FIG. 7 shows that the xylitol diffluent is a single peak in HPLC in the calcium type cation resin simulated moving bed.

FIG. 8 shows that the arabinose-saccharide diffluence's HPLC print in the calcium type cation resin simulated moving bed. (1, arabinose, 2, hetero saccharide, 3, hetero saccharide).

FIG. 9 is second HPLC chromatographic separation of the arabinose diffluent in HPLC in the ammonium type cation resin. The main peak is arabinose.

### DETAILED DESCRIPTION OF THE PREFERRED EMBODIMENTS

### Embodiment 1

Operation 1

Weigh 3 kg sugar cane bagasse which has been washed and dried, and according to solid-to liquid ratio (w/v) 1:7 to add H2SO4 (2.5%, w/w), 120° C., 2 h, then centrifugated and collect the filtrate, the filter residue is washed by water, and the water is then mixed to the filtrate, the PH of the mixed filtrate is adjusted to 3 by solid calcium, filter to remove residue, then sugar cane bagasse hemicellulose hydrolysates is obtained. The hydrolysates contained 3% reducing sugar, the xylose is 80% in total sugar.

Operation 2

Weigh 3 kg dried and crushed corn core, and according to solid-to liquid ratio (w/v) 1:7 to add H2SO4 (2%, w/w), 120° C., 2 h, then centrifugated and collect the filtrate, the PH of the mixed filtrate is adjusted to 3 by solid calcium, filter to remove residue, then corn core hemicellulose hydrolysate is obtained. The hydrolysate contains 5% total sugar, herein the xylose is 60% in total sugar.

Operation 3

CCTCC NO:M206098 is slant inoculated to liquor seed medium, cultured in 200 rmp, 30° C. for 12 h, then inoculated into sugar cane bagasse hemicellulose hydrolysates of operation 1 according to 15% (v/v) inoculation, fermented in 200 rmp, 33° C. for 45 h, after centrifugation, the supernatant, i.e. the sugar cane bagasse hemicellulose hydrolysates detoxified by CCTCC NO:M206098 is obtained.

(or in same conditions, to use CCTCC NO:M206097 to treat sugar cane bagasse hemicellulose hydrolysates of operation 1, then sugar cane bagasse hemicellulose hydrolysates detoxified by CCTCC NO:M206097 is obtained).

Operation 4

is detoxified sugar cane bagasse hemicellulose hydrolysates in operation 3 is concentrated to 150 g/L xylose under vacuum, PH is adjusted to 6 by ammonia water, add 5 g/L yeast extract and inoculate Candida tropicalis) CCTCC NO:M205067, (v/v) inoculation, fermented in 200 rpm, 33° C. Compared the HPLC detecting results (table 3), the hemicellulose hydrolysates after detoxification used for xylitol fermentation, both the producing rate and the conversion are more than doubled.

TABLE 3
effect of the bio-detoxification for xylitol ferment
(original xylose, 150 g/L; ferment time 61.5 h)

| | | Concen- | | Xylitol | Dry |
|---|---|---|---|---|---|
| | Xylose | tration | Xylitol | production | cells |
| | utilization | of xylitol | conversion | rate | density |
| Detocification | rate (%) | (g/L) | (g/g) | (g/L · h) | (g/L) |
| Control | 77.5 | 63.2 | 0.54 | 1.03 | 14.6 |
| S-7 | 90 | 125.0 | 0.93 | 2.03 | 14.3 |
| Lj-3 | 93.0 | 124.0 | 0.89 | 2.01 | 13.6 |

Operation 5

hemicellulose hydrolysates in operation 1 is concentrated to 120 g/L xylose under vacuum, PH is adjusted to 5 by ammonia water, one group is inoculated in CCTCC NO:M206098 and CCTCC NO:M 205067 in same quantity which are collected by centrifugation, the other group is inoculated in CCTCC NO:M206097 and CCTCC NO:M 205067 in same quantity. All their total inoculation is drying cell 50 g/L. 200 rpm shaking ferment for 30 h. The result (table 4) shows that the ferment and detoxification can improve the concentration, production rate and conversion rate of the ferment product,

TABLE 4
comparison of the ferment with detocxification of hemicellulose
hydrolysates and the direct ferment of hemicellulose hydrolysates
(original xylose, 120 g/L; ferment time 30 h)

| | | Terminal | Xylitol | | Xylitol |
|---|---|---|---|---|---|
| | Remain | drying | concen- | Conversion | production |
| Combination | xylose | cells | tration | rate | rate |
| mode | (g/L) | (g/L) | (g/L) | (g/g) | (g/L · h) |
| CCTCC | 33.5 | 49.3 | 50.98 | 0.59 | 1.7 |
| NO: M205067 | | | | | |
| (control) | | | | | |
| CCTCC | 10.7 | 52.5 | 77.6 | 0.71 | 2.6 |
| NO: M205067 | | | | | |
| and | | | | | |
| CCTCC | | | | | |
| NO: M206097 | | | | | |
| CCTCC | 8.6 | 55.0 | 81.3 | 0.73 | 2.7 |
| NO: M205067 | | | | | |
| and | | | | | |
| CCTCC | | | | | |
| NO: M206098 | | | | | |

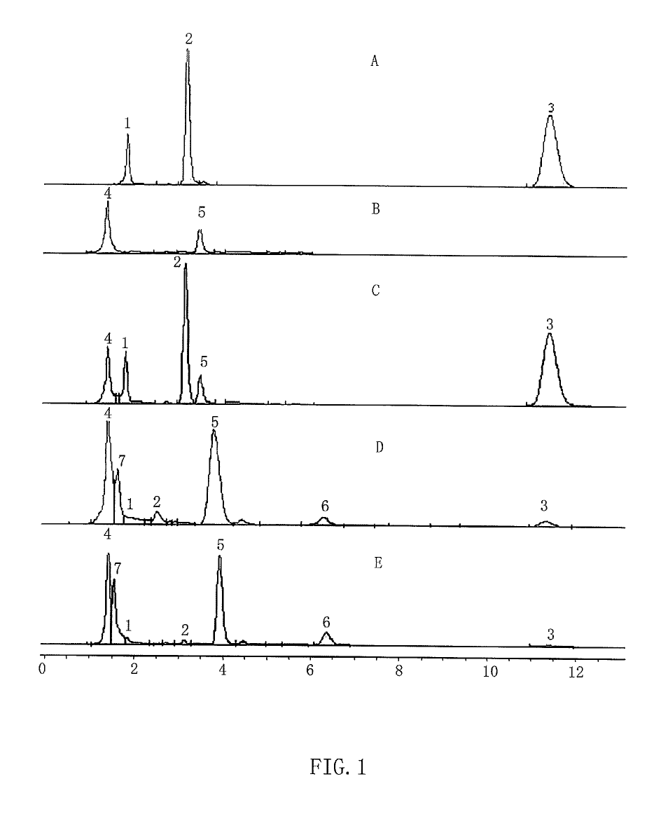

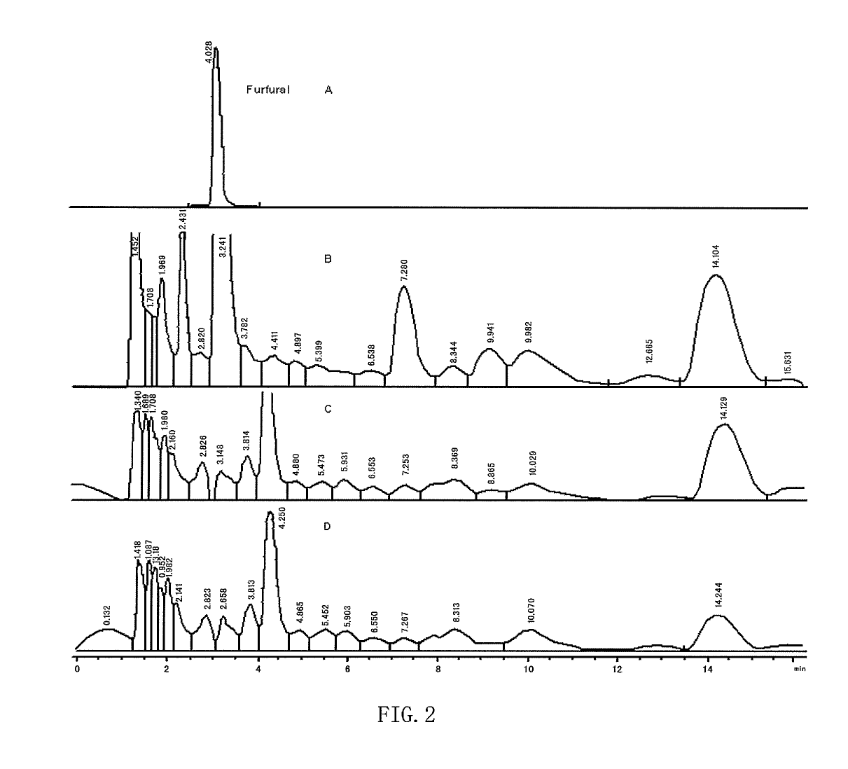

Referring to FIG. 1, it shows the change of HPLC of the inhibitor degraded by stains. Parameter: (A) inhibitor, (B) medium, (C) medium added with inhibitor, (D) medium containing inhibitor fermented by CCTCC NO:M206097, (E) medium containing inhibitor fermented by CCTCC NO:M206098. Peaks and compounds: 1 acetic acid, 2 furfural, 3, guaiacol, 4, 5 components of medium, 6, degraded product of furfural, 7 degraded product of guaiacol. FIG. 2 is a HPLC fingerprint before and after the bio detoxification of the sugar cane bagasse hemicellulose hydrolysates (270 nm). Parameter: A furfural sample; B sugar cane bagasse hemicellulose hydrolysates; C, sugar cane bagasse hemicellulose hydrolysates detoxified by CCTCC NO:M206098; D, sugar cane bagasse hemicellulose hydrolysates detoxified by CCTCC NO:M206097.

TABLE 5
the concentration change of some compounds in the sugar cane
bagasse hemicellulose hydrolysates after detoxification
(270 nm)

| | | | | CCTCC NO: M206098 | | CCTCC NO: M206097 | |
|---|---|---|---|---|---|---|---|
| | | | | Peak | | Peak | |
| | Peak | | Peak area | area | Decrease | area | Decrease |
| Peak | appearing | | before | after | rate | after | rate |
| number | time (min) | compounds | detoxification | detoxification | (%) | detoxification | (%) |
| 1 | 1.4 | Un | 1506.6 | 364.9 | 75.8 | 0 | 100 |
| 2 | 1.8 | Un | 138.6 | 0 | 100 | 0 | 100 |
| 3 | 1.9 | Un | 575.3 | 384.8 | 33.1 | 373.8 | 35 |
| 4 | 2.4 | Un | 622.0 | 0 | 100 | 0 | 100 |
| 5 | 3.2 | furfural | 10747.1 | 467.4 | 95.7 | 329.2 | 96.9 |
| 6 | 7.2 | Un | 1195.5 | 338.0 | 71.7 | 162.2 | 86.4 |
| 7 | 9.1 | Un | 500.5 | 234.2 | 53.2 | 0 | 100 |
| 8 | 10.0 | Un | 837.3 | 625.9 | 25.3 | 509.1 | 39.2 |
| 9 | 41.1 | Un | 2692.5 | 2078.2 | 22.8 | 860.3 | 68.0 |

### Embodiment 2

Operation 1

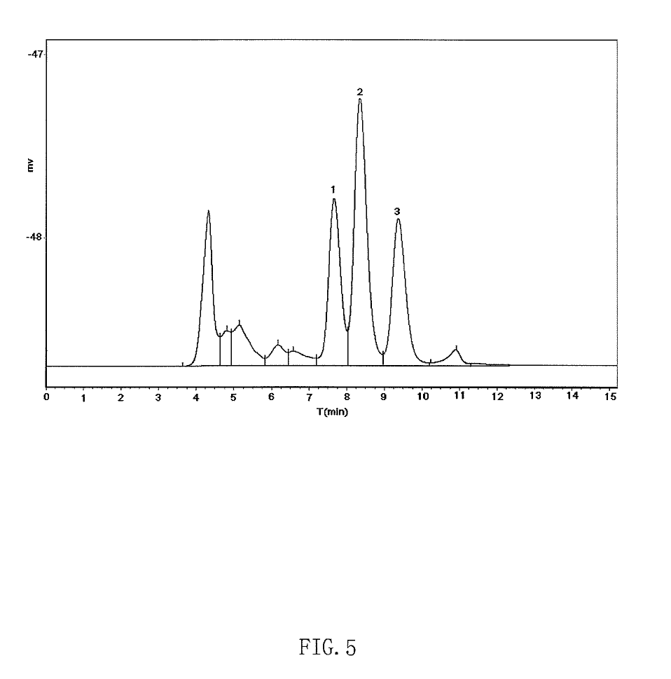

Add 160 L water into 40 kg corn fiber and then boiled. Remove the liquor and the residue is washed one time by water, after drying, add 2% (w/w) H2SO4 80 L, 125° C., 2 h, then filtrate and collect the filtrate, the PH of the filtrate is adjusted to 3 by calcium carbonate, filter to remove residue, then hydrolysates add 1 kg activity carbon to adsorb for 30 minutes and then remove the carbon, and the filtrate pass cation and anion resin, transparent syrup is obtained and then concentrated to a required concentration in reduced pressure condition, The syrup contains glucose, xylose and arabinose about the ratio 1:2:1 (FIG. 5).

Operation 2

Dried sugar cane bagasse 40 kg, added in 2.4% (w/w) H2SO4 280 L, 125° C. 2 h, the other steps are the same as operation 1, syrup obtained by sugar cane bagasse hemicellulose hydrolysates contains glucose, xylose and arabinose about the ratio 1:10:1.

Operation 3

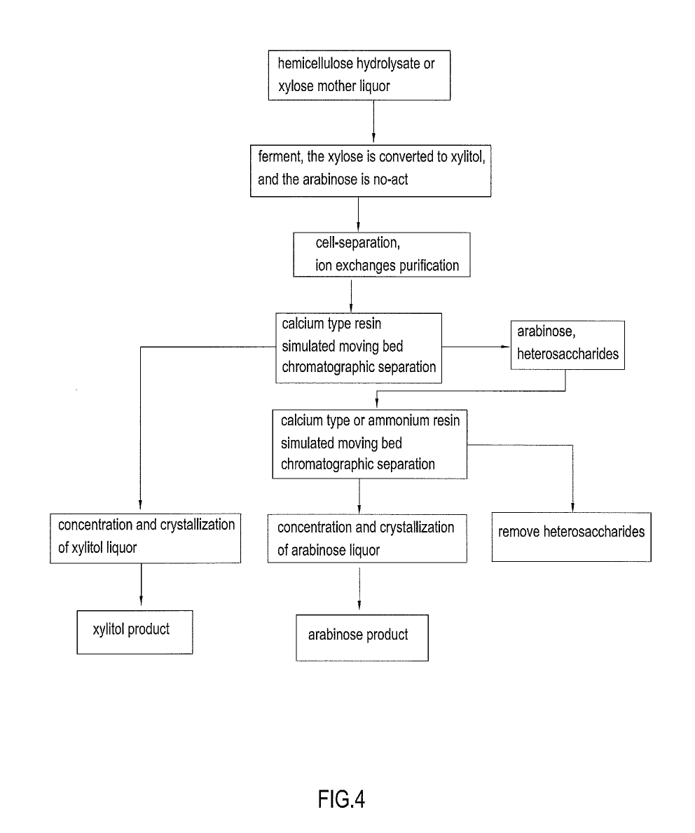

Add 100 g hot water extract from rice bran into each litre corn fiber hemicellulose hydrolysates in operation 1, and adjust to total sugar 250 g/L, herein xylose is about 120 g/L, arabinose is about 80 g/L, 110° C. sterilization for 10 minutes. Then fill 3 L hydrolysates into 5 L ferment pot (Biotech-5BG; Shanghai Baoxin Bioengineering. Equipment, Shanghai, China), and inoculated with Candida tropicalis CCTCC M 205067, inoculation is 5% (v/v), aeration 1 vvm, 300 rpm, 33° C., 30 h, the xylose is converted into xylitol, the arabinose is no-react (FIG. 4). After ferment, centrifuge to collect cells, and the cells are added to fresh medium and refill into ferment pot. The medium filled into the first ferment volume, and maintain the same aeration and stirring rate. The results of the continuous five ferments are shown in table 7.

TABLE 6
selectively convert the corn fiber hemicellulose
hydrolysates into xylitol

| | | Original material | | Ferment products | |
|---|---|---|---|---|---|
| | | combination | | combination | |
| Cell | Ferment | xylose | | xylitol | arabinose |
| cycle | time | (g/L) | arabinose (g/L) | (g/L) | (g/L) |
| 1 | 30 | 118.0 | 84.3 | 100.7 | 82.6 |
| 2 | 26 | 123.5 | 86.3 | 103.5 | 86.3 |
| 3 | 18 | 120.2 | 85.4 | 109.8 | 82.5 |
| 4 | 19 | 122.8 | 86.3 | 110.5 | 85.3 |
| 5 | 18 | 125.0 | 88.7 | 110.6 | 88.5 |

Operation 4

Xylose crystallization mother liquor of xylose mill is fermented by pre-cultured strains in 3T ferment pot, then centrifugated by industrial centrifuge to collect stain cells, and the other conditions are the same as operation 3. The results of the continuous five ferments are shown in table 8.

TABLE 7
Selectively convert the xylose crystallization mother liquor
into xylitol

| | | Original material | | Ferment products | |
|---|---|---|---|---|---|
| | | combination | | combination | |
| Cell | Ferment | xylose | | xylitol | arabinose |
| cycle | time | (g/L) | arabinose (g/L) | (g/L) | (g/L) |
| 1 | 25 | 120.5 | 45.5 | 108.4 | 45.3 |
| 2 | 20 | 119.5 | 44.3 | 112.5 | 44.2 |
| 3 | 19 | 123.6 | 46.5 | 119.4 | 46.3 |
| 4 | 17 | 120.4 | 45.3 | 114.9 | 45.3 |
| 5 | 17 | 124.6 | 46.8 | 116.5 | 46.2 |

Operation 5

10 L fermented liquor of operation 3 is ultrafiltrated by a unitrafiltration membranes with molecular weight cut off 5 Kdal to remove the proteins, then pass though cation-anion-cation-anion columns in turn to desalt and decolor (cation resin 001><7; anion resin D301, Nankai university, china), thus obtained transparent xylitol-arabinose pure liquor. The liquor is then concentrated into 60% soluble material in reduced pressure condition.

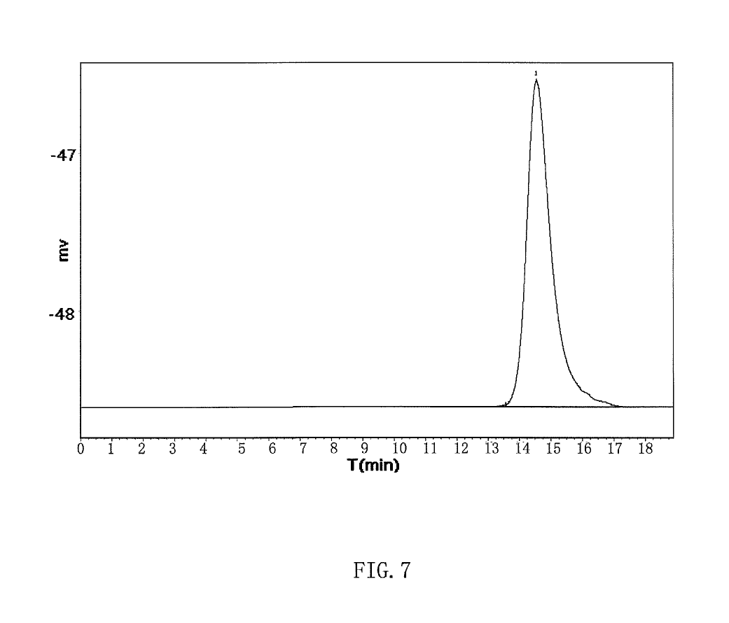

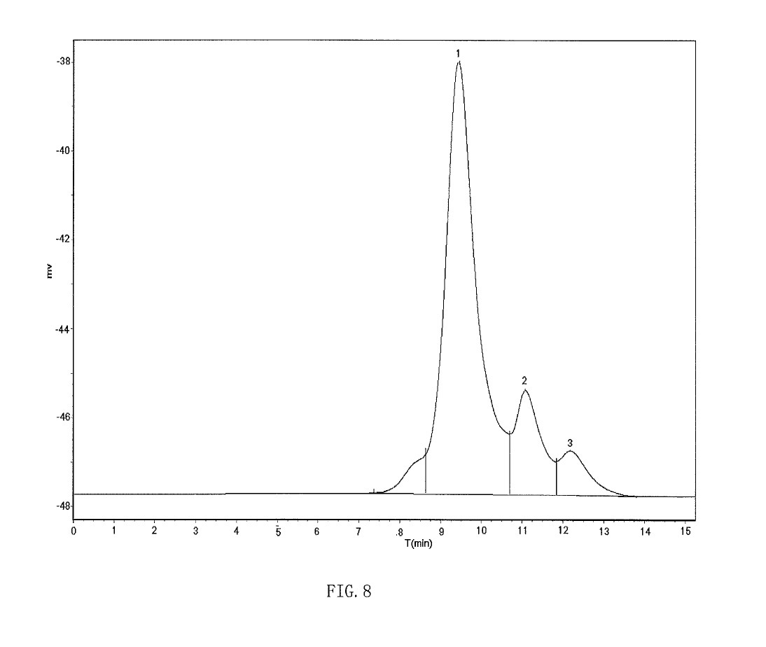

Concentrated liquor is first chromatographic separation by a simulated moving bed system with 20 columns filled by calcium resin(AMBERLITE CR1320Ca), the separation temperature is 60° C., rate of feed is 5 ml/min, pure water washing rate is 25 ml/min. after balance, the flow is consisted by: 1 xylitol portion with more than 99% xylitol (FIG. 7), concentration is 12-13%; 2. arabinose-heterosaccharides portion, total soluble solid 5-7%, herein the arabinose is 55-60% (FIG. 8), the other impurity 30-40%. The xylitol liquor is directly concentrated and crystallized to obtain crystalline xylitol, the arabinose-saccharides portion is to be purified next.

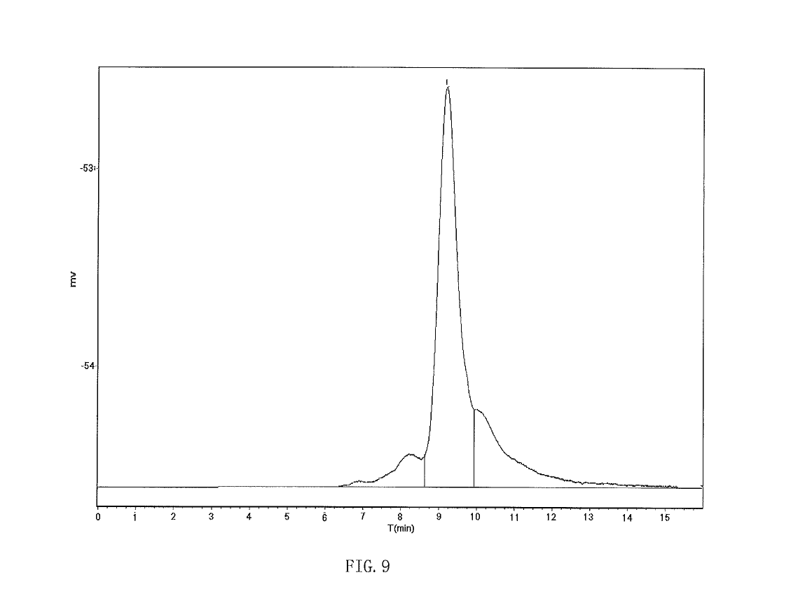

The arabinose-saccharides liquor is concentrated in soluble solid is 60% in reduced pressure, then AMBERLITE CR1320Ca resin is converted into ammonium type by ammonium salts, the second chromatographic separation is processed by the same simulated moving bed system. separation temperature is 30° C., rate of feed is 3 ml/min, pure water washing rate is 23 ml/min. After balance, the arabinose portion is collected, herein the purity of the arabinose is rise to more than 85% (FIG. 9) from feed, after being concentrated, crystalline arabinose with purity more than 99% is obtained.

What is claimed is:

1. A method of producing xylitol and arabinose at same time from hemicellulose hydrolysates, comprising:
S1, preparing hemicellulose hydrolysate;
S2, concentrate the hemicellulose hydrolysate under vacuum to a content that the xylose concentration is 50-150 g/L, adjust the PH to 3-7, by centrifugation or filtration to remove the residue, then filtrate of hemicellulose hydrolysate is obtained;
S3, the filtrate of hemicellulose hydrolysate is inoculated in both Issatchenkia orientalis S-7 and Candida tropicalis CCTCC NO:M205067, fermented until the xylose is completely consumed;
S4, remove the cells, after being purified by iron exchanges, and chromatographic separation by calcium type cation resin, purified xylitol diffluence is obtained;
S5, after the purified xylitol diffluence, the arabinose diffluence is obtained.

2. The method of producing xylitol and arabinose at same time from hemicellulose hydrolysates according to claim 1 further comprising:
S6, centrifugate the yeast cells and re-used in fresh filtrate of hemicellulose hydrolysate, continue to ferment and recycle the cells until the cells can not be used.

3. The method of producing xylitol and arabinose at same time from hemicellulose hydrolysates according to claim 1, wherein said Issatchenkia orientalis S-7 is the strain conserved in CCTCC, the conservation number is CCTCC NO:M206098.

4. The method of producing xylitol and arabinose at same time from hemicellulose hydrolysates according to claim 1, wherein the hemicellulose hydrolysate is obtained by material rich in fibre being hydrolyzed by diluted acid or enzymes.

5. The method of producing xylitol and arabinose at same time from hemicellulose hydrolysates according to claim 4, wherein the material is sugar cane bagasse, straws of crops, corn fiber or corn core.

6. The method of producing xylitol and arabinose at same time from hemicellulose hydrolysates according to claim 4, wherein the Issatchenkia orientalis S-7 and Candida tropicalis CCTCC NO:M205067 are mixed by same volume, and the mixture is inoculated into the filtrate of hemicellulose hydrolysate according to inoculation of 1˜10% (v/v).

7. The method of producing xylitol and arabinose at same time from hemicellulose hydrolysates according to claim 1, wherein the purified xylitol diffluence in step S4 is concentrated and crystallized to obtain crystalline xylitol.

8. The method of producing xylitol and arabinose at same time from hemicellulose hydrolysates according to claim 1, wherein the arabinose diffluence in step S5 is chromatographic separation by ammonium type is cation resin, and then concentrated and crystallized to obtain crystalline arabinose.

9. A method of producing xylitol and arabinose at same time from hemicellulose hydrolysates, comprising:
S1, preparing hemicellulose hydrolysate;
S2, concentrate the hemicellulose hydrolysate under vacuum to a content that the xylose concentration is 50-150 g/L, adjust the PH to 3-7, by centrifugation or filtration to remove the residue, then filtrate of hemicellulose hydrolysate is obtained;
S3, the filtrate of hemicellulose hydrolysate is inoculated in both Issatchenkia occidentalis LJ-3 and Candida tropicalis CCTCC NO:M205067, fermented until the xylose is completely consumed;
S4, remove the cells, after being purified by iron exchanges, and chromatographic separation by calcium type cation resin, purified xylitol diffluence is obtained;
S5, after the purified xylitol diffluence, the arabinose diffluence is obtained.

10. The method of producing xylitol and arabinose at same time from hemicellulose hydrolysates according to claim 9 further comprising:
S6, centrifugate the yeast cells and re-used in fresh filtrate of hemicellulose hydrolysate, continue to ferment and recycle the cells until the cells can not be used.

11. The method of producing xylitol and arabinose at same time from hemicellulose hydrolysates according to claim 9, wherein said Issatchenkia occidentalis LJ-3 is the strain conserved in CCTCC, the conservation number is CCTCC NO:M206097.

12. The method of producing xylitol and arabinose at same time from hemicellulose hydrolysates according to claim 9, wherein the hemicellulose hydrolysate is obtained by material rich in fibre being hydrolyzed by diluted acid or enzymes.

13. The method of producing xylitol and arabinose at same time from hemicellulose hydrolysates according to claim 12, wherein the material is sugar cane bagasse, straws of crops, corn fiber or corn core.

14. The method of producing xylitol and arabinose at same time from hemicellulose hydrolysates according to claim 9, wherein the Issatchenkia occidentalis LJ-3 and Candida tropicalis CCTCC NO:M205067 are mixed by same volume, and the mixture is inoculated into the filtrate of hemicellulose hydrolysate according to inoculation of 1˜10% (v/v).

15. The method of producing xylitol and arabinose at same time from hemicellulose hydrolysates according to claim 9, wherein the purified xylitol diffluence in step S4 is concentrated and crystallized to obtain crystalline xylitol.

16. The method of producing xylitol and arabinose at same time from hemicellulose hydrolysates according to claim 9, wherein the arabinose diffluence in step S5 is chromatographic separation by ammonium type cation resin, then is concentrated and crystallized to obtain crystalline arabinose.

17. A method of producing xylitol and arabinose at same time from hemicellulose hydrolysates, comprising:
S1, preparing hemicellulose hydrolysate;
S2, concentrate the hemicellulose hydrolysate under vacuum to a content that the xylose concentration is 50-150 g/L, adjust the PH to 3-7, by centrifugation or filtration to remove the residue, then filtrate of hemicellulose hydrolysate is obtained;
S3, the filtrate of hemicellulose hydrolysate is inoculated in Issatchenkia orientalis S-7 to detoxification, then inoculated in Candida tropicalis CCTCC NO:M205067, fermented until the xylose is completely consumed;
S4, remove the cells, after being purified by iron exchanges, and chromatographic separation by calcium type cation resin, purified xylitol diffluence is obtained, the purified xylitol diffluence is concentrated and crystallized to obtain crystalline xylitol;
S5, after the purified xylitol diffluence, the arabinose diffluence is obtained, the arabinose diffluence is chromatographic separation by ammonium type cation resin, then is concentrated and crystallized to obtain crystalline arabinose.
